(19) United States
(12) Patent Application Publication
(10) Pub. No.: US 2020/0268617 A1
(43) Pub. Date: Aug. 27, 2020

(54) MINERALIZED TISSUE ADHESIVE/FILLER COMPOSITION COMPRISING CROSSLINKED OSTEOPONTIN

(71) Applicant: THE ROYAL INSTITUTION FOR THE ADVANCEMENT OF LEARNING / MCGILL UNIVERSITY, Montreal (CA)

(72) Inventors: François BARTHELAT, Boulder, CO (US); Sacha CAVELIER, Montreal (CA); Ahmad KHAYER DASTJERDI TOROGHI, Toronto (CA); Marc D. MCKEE, Montreal (CA)

(21) Appl. No.: 16/792,423

(22) Filed: Feb. 17, 2020

Related U.S. Application Data
(60) Provisional application No. 62/808,944, filed on Feb. 22, 2019.

(51) Int. Cl. A61K 6/884; A61K 6/20; A61L 24/10; A61K 6/30; A61L 27/22

(52) U.S. Cl. CPC A61K 6/884; A61K 6/20; A61L 2430/02; A61K 6/30; A61L 27/227; A61L 24/108

(57) ABSTRACT

The present disclosure concerns a mineralized tissue adhesive or filler composition comprising an intermolecular crosslinked osteopontin protein for treating an exposed surface of a mineralized tissue. The mineralized tissue adhesive or filler composition can be added directly on the exposed surface of the mineralized tissue or on a layer of monodisperse OPN protein which has been deposited on the exposed surface of the mineralized tissue. The present disclosure also includes methods and kits of using the mineralized tissue adhesive or filler composition especially in dental applications. The present application also provides dental coatings, fillings, bondings and veneers comprising the mineralized tissue adhesive or filler composition described herein.

### CROSS-REFERENCE TO RELATED APPLICATIONS

The present application claims priority to U.S. provisional patent application Ser. No. 62/808,944 filed on Feb. 22, 2019 and herewith incorporated in its entirety.

### TECHNOLOGICAL FIELD

The present disclosure relates to a composition capable of adhering to mineralized tissue such as bones, cartilage and teeth, for treating same.

### BACKGROUND

Bone, as a hierarchically organized material, has a remarkably high combination of strength, stiffness and toughness, all of which are critical to the functions of the skeleton in support, protection and resistance to impacts. Bone is composed primarily of calcium phosphate-based mineral, collagen, noncollagenous proteins, small proteoglycans and water organized over its many levels of hierarchy [1-3]. By weight, the majority of protein found in bone is collagen type I which forms an extensive fibrillar network in the extracellular matrix. However, on a molar basis, noncollagenous proteins collectively are present in similar amounts as collagen. The noncollagenous proteins generally accumulate between the collagen fibrils in the so-called interfibrillar compartment [4, 5] where it appears plausible that they may serve as a toughening “binder” within the bulk extracellular matrix [6]. Likewise, the prominent accumulation of osteopontin (OPN) at interfacial cement lines [7, 8] arising from the reversal of bone resorption to bone formation during its remodeling cycle, where new bone is deposited onto older bone, also points to a potential adhesive role. Indeed, many interfaces are present at the different levels of hierarchy in bones, and these are considered to be the initiators of powerful toughening mechanisms [4].

In teeth, which is composed of three distinct tissues (enamel, dentin and cementum), tooth dentin and tooth cementum have similar composition, structure and properties as bone. Erupted tooth enamel, having a different developmental origin (an epithelial origin) from other tooth tissues, consists predominantly of mineral, and very little protein, and thus it is very hard (but brittle), and less tough than bone, dentin and cementum because of this lower protein content.

In bone, intermolecular bonds within and between collagen fibrils, and also involving noncollagenous proteins, and collagen organization itself (i.e. between lamellae of the osteons), and the bonding also occurring across cement lines delimiting osteons (also called Haversian systems), can all generally be considered as “weak” interfaces which channel deformation [9] and deflect cracks [10] (FIG. 1). Other bonds also form between the organic and inorganic (mineral) phases. During the forces incurred through skeletal function, cement lines/planes are a site of twisting and other deformations, where debonding, frictional pullout, crack bridging and crack deflection all may occur [10, 11]. Previously, it has been speculated that OPN is responsible for matrix/mineral adhesion within cement lines in bone, and there may act as an interfacial adhesion promoter in mineralized tissues (between newly formed and older bone during remodeling) [8].

In teeth, which do not have cement lines related to remodeling sites as in bone, OPN is found predominantly at the interface between tooth dentin and cementum, and also throughout the bulk tissue of each of these tissues. Being present also at tooth interfaces is consistent with a role as an adhesion molecule [45, 46].

In bone, weaker interfaces can deflect cracks (in interfibrillar matrix and cement lines) and are rich in noncollagenous proteins (and their extended networks) [4]. Prominent at these sites, OPN resides as a flexible, extended, intrinsically disordered phosphoprotein having a remarkably high negative charge that arises from an abundance of acidic amino acid residues (Asp and Glu) and the presence of many phosphorylated serine residues distributed along the full length of the protein [12]. While OPN is found in a wide variety of tissues and biological fluids from various species [13, 14], its prominent abundance in bone extracellular matrix involves roles in regulating mineralization [15], providing cell adhesion ligands for integrin receptors [16], and generating mechanical performance enhancement, the latter observation supported by experiments showing that an absence of this protein (in OPN-deficient “knockout” mice) has a negative impact on the toughness of bone at the macroscale [17].

Cement lines/planes are not the only interfaces in bone in which OPN likely plays a mechanical role. In the molecular nano-environment of the interfibrillar compartment of the extracellular matrix of bone (and also of tooth dentin and cementum), there are also interactions between the mineral crystallites and the abundant organic matrix molecules residing there, where specific deformation and toughening mechanisms would be expected to occur at the nanometer scale, in particular between collagen fibrils, other noncollagenous proteins and proteoglycans, and mineral nano-crystals, all of which in principle shear past one another under skeletal loading (FIG. 1A). Indeed, fracture-toughness tests on the weaker bones of knockout mice lacking OPN have shown the importance of this protein in toughening bone [17]. Relevant to a toughening mechanism for bone involving OPN is that this protein binds strongly both ionic calcium and mineral/crystal lattice calcium atoms through its overall negative charge and specific acidic peptide motifs that include phosphorylation sites [7]. Moreover, OPN also binds to osteocalcin, another abundant noncollagenous bone protein [18]. With regard to the interaction between these two proteins, OPN forms a complex with osteocalcin as shown in FIG. 2B [4] that is capable of dissipating energy when two adjacent collagen fibrils are under shear load (FIG. 1C). The high negative charge of OPN allows for extensive binding to positively charged calcium ions to form sacrificial bonds (FIG. 1B) that break under shear load and allow energy-dissipating extension (without rupture) of the OPN molecule—such a toughening mechanism is depicted in FIG. 1C. Such sacrificial bonds can reform rapidly in the presence of calcium to allow nanoscale-level “healing” and repeated energy dissipation [4, 6]. In principle, this particular process ends when the OPN molecule is fully stretched, but it can be repeated over multiple cycles of loading [6].

It would be desirable to provide an adhesive or a filler capable of adhering to and treating a mineralized tissue to limit the degradation of the tissue, maintain the integrity of the tissue or restore the integrity of the tissue. In some embodiments in which the mineralized tissue is a tooth, it would be desirable to provide the tooth with an adhesive or filler for limiting or preventing the onset of cavities (caries), that may even assist in remineralizing the defect, and/or limiting cracking and crack propagation following excessive forceful mastication causing microcracks, or after other trauma to teeth.

### BRIEF SUMMARY

The present disclosure provides an adhesive or a filler composition for the treatment of an exposed surface of a mineralized tissue. The adhesive or filler composition comprises intermolecular crosslinked osteopontin (OPN) protein.

In a first aspect, the present disclosure provides a method of treating an exposed surface of a mineralized tissue. The method comprises contacting a mineralized tissue adhesive or filler composition having intermolecular crosslinked osteopontin (OPN) protein directly with (i) the exposed surface or (ii) a first layer of monodisperse OPN protein directly deposited on the exposed surface. In an embodiment, the exposed surface comprises at least one crack and the method further comprises filing the at least one crack. In another embodiment, the method further comprises contacting the mineralized tissue adhesive or filler composition directly with (i) a second surface or (ii) a second layer of monodisperse OPN protein directly deposited on the second surface so as to adhere the first surface to the second surface. In an embodiment, the second surface is a mineralized tissue (such as a tissue graft) or an artificial surface (such as a dental prosthetic). In some embodiments, the method further comprises depositing monodisperse OPN protein on the exposed surface and/or the second surface to form the first layer and/or the second layer of monodisperse OPN protein. In yet additional embodiments, the method further comprises providing the mineralized tissue adhesive or filler composition. In such embodiment, the method comprises crosslinking monodisperse OPN protein with a crosslinking agent to provide the mineralized tissue adhesive or filler composition. The crosslinking agent can be a transglutaminase (such as transglutaminase 2) and/or glutaraldehyde. In some embodiments, the mineralized tissue is a tooth and as such, the exposed surface can comprise enamel, dentin and/or cementum. In other embodiments, the mineralized tissue is a bone. In some embodiments, the mineralized tissue is in a subject.

In a second aspect, the present disclosure provides a kit for making and using a mineralized tissue adhesive or filler composition to treat an exposed surface of a mineralized tissue. The kit comprises a monodisperse osteopontin (OPN) protein composition; a crosslinking agent; and instructions for making and using the mineralized tissue adhesive or filler composition. The instructions include preparing the mineralized tissue adhesive or filler composition by crosslinking, with the crosslinking agent, the monodisperse OPN protein composition, wherein the mineralized tissue adhesive or filler composition comprises intermolecular crosslinked OPN protein. The instructions can optionally include preparing a monodisperse solution of OPN protein from the monodisperse OPN protein composition. The instructions can optionally include optionally depositing a first layer of monodisperse OPN protein on the exposed surface of the mineralized tissue to form a first layer of monodisperse OPN protein. The instructions can optionally include depositing a second layer of monodisperse OPN protein on a second surface to form a second layer of monodisperse OPN protein. The instructions include directly contacting the mineralized tissue adhesive or composition on (i) the exposed surface of the mineralized tissue or on the first layer of monodisperse OPN protein and optionally on (ii) the second surface or on the second layer of monodisperse OPN protein. In an embodiment, the kit further comprises an applicator for contacting the mineralized tissue adhesive or filler composition on the exposed surface or on the first layer of monodisperse OPN protein. In still another embodiment, the applicator can also be used for contacting the monodisperse solution of OPN protein on the exposed surface or the second surface. In an embodiment, the crosslinking agent is a transglutaminase (such as, for example, transglutaminase 2) and/or glutaraldehyde. In yet another embodiment, the mineralized tissue is a tooth and the tooth surface can comprise enamel, dentin and/or cementum. In still another embodiment, the kit further comprises a first container for the monodisperse OPN protein composition and a second container for the crosslinking agent.

According to a third aspect, the present disclosure provides dental protective coating comprising the mineralized tissue adhesive or filler composition defined herein.

According to a fourth aspect, the present disclosure provides a dental filling comprising the mineralized tissue adhesive or filler composition defined herein.

According to a fifth aspect, the present disclosure provides a dental bonding or veneer comprising the mineralized tissue adhesive or filler composition defined herein.

According to a sixth aspect, the present disclosure provides a dental adhesive comprising the mineralized tissue adhesive or filler composition defined herein.

### BRIEF DESCRIPTION OF THE DRAWINGS

Having thus generally described the nature of the invention, reference will now be made to the accompanying drawings, showing by way of illustration, a preferred embodiment thereof, and in which:

FIGS. 1A to 1C (prior art) represent the extracellular matrix organization in bone.

(FIG. 1A) Four of the levels of hierarchy in bone (adapted from [4]).

(FIG. 1B) Molecular interactions involving osteopontin (OPN, red) and osteocalcin (OCN, blue) between collagen type I fibrils (adapted from [4]).

(FIG. 1C) Energy dissipation mechanisms involving OPN when two fibrils are subjected to shear load (adapted from [4]).

FIGS. 2A to 2D (prior art) provide graphical representations of the crosslinking of OPN.

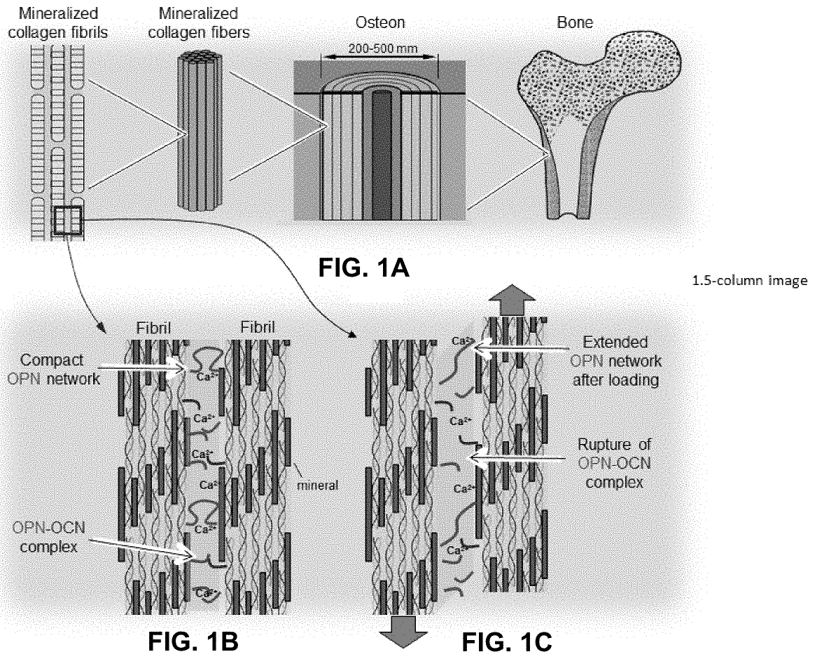

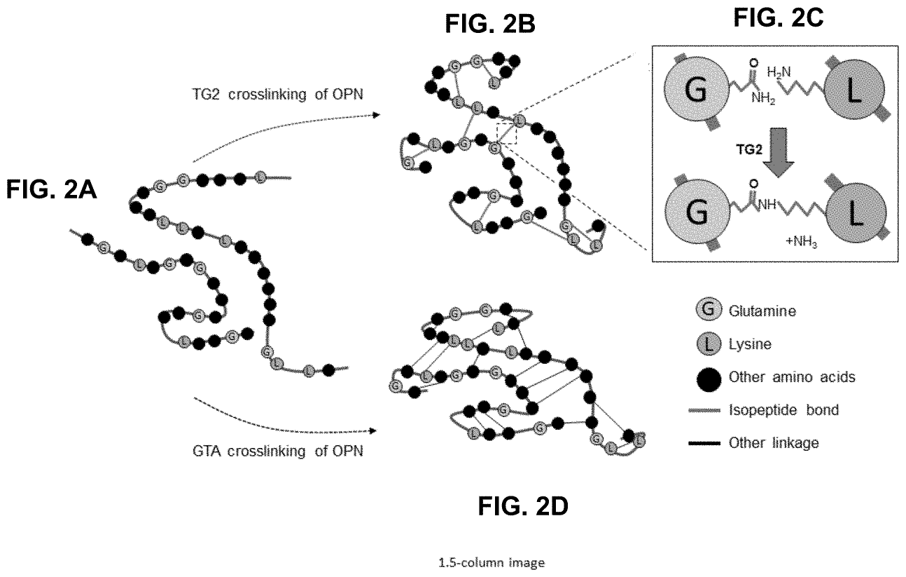

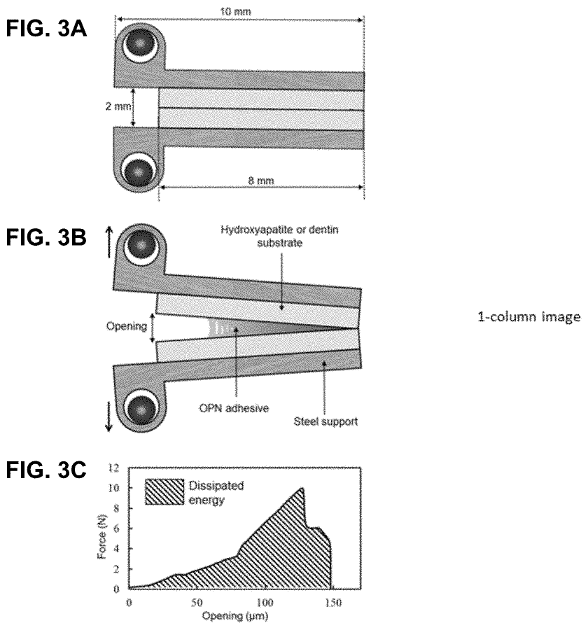

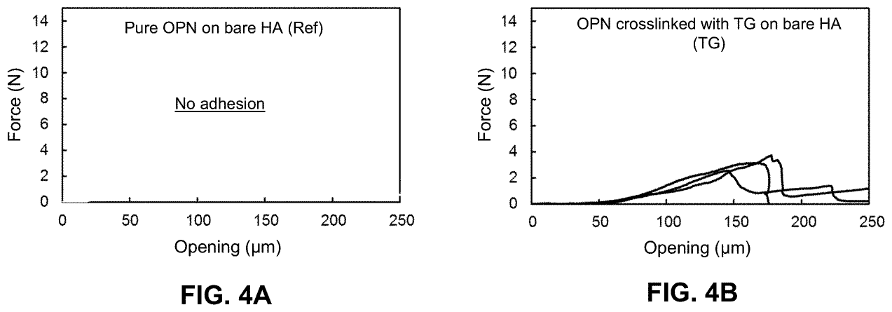

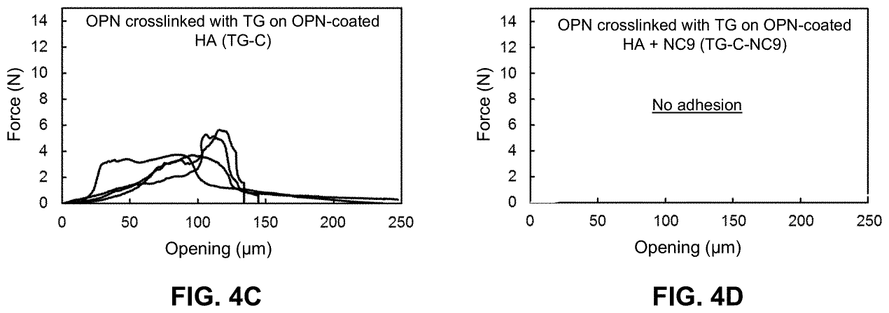

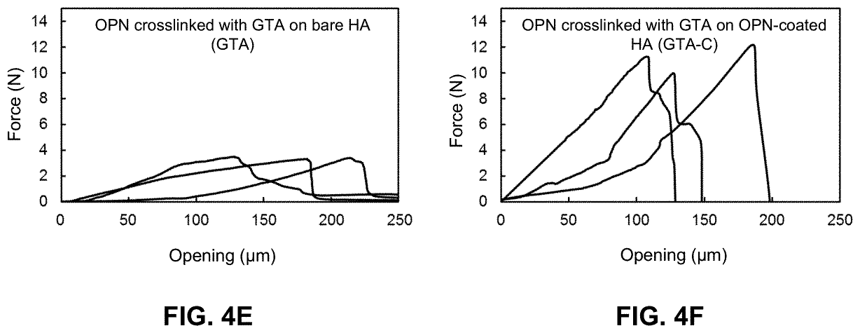

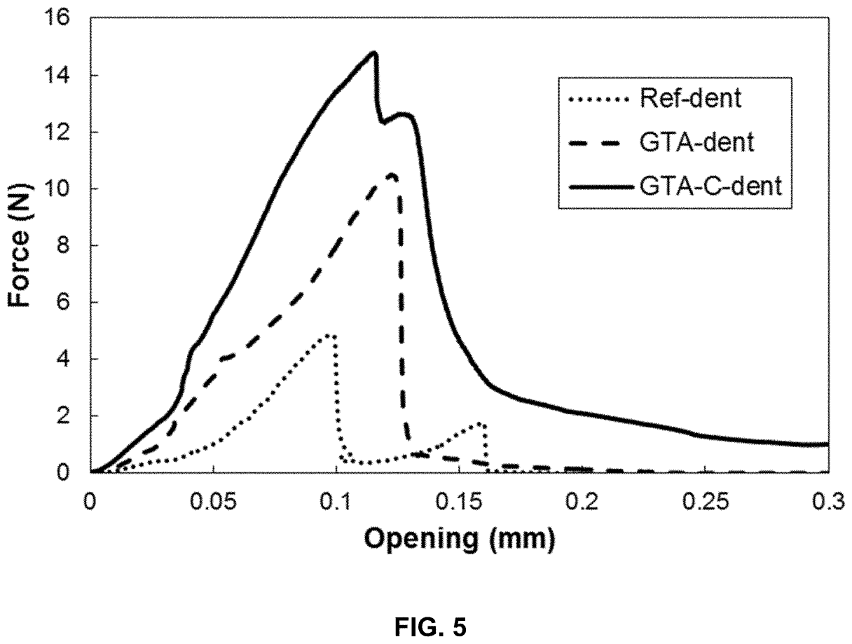

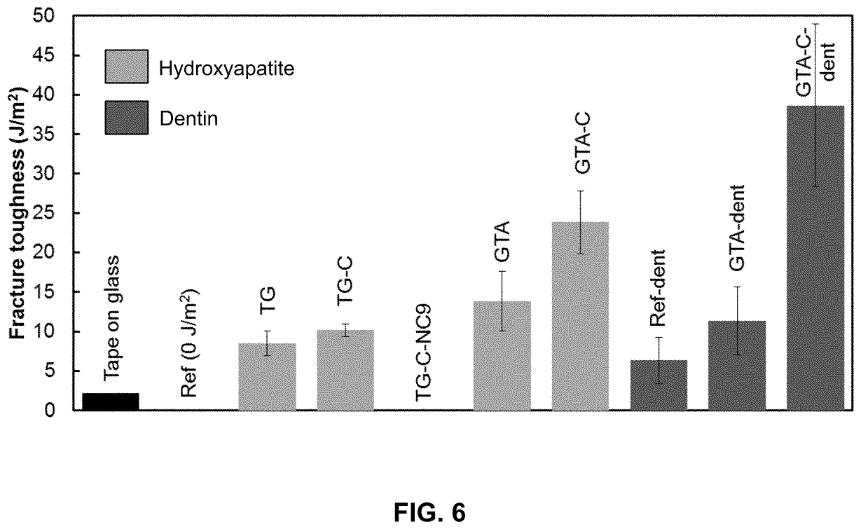

(FIG. 2A) Two simplified OPN molecules having glutamine and lysine for crosslinking reactions, and other amino acids.

(FIG. 2B) TG2 crosslinking of two simplified OPN molecules. γ-glutamyl-ε-lysine (isopeptide) bonds are formed between glutamine and lysine amino acids both intra- and inter-molecularly.

(FIG. 2C) Chemical reaction between glutamine and lysine in the presence of TG2 leading to a γ-glutamyl-ε-lysine bond.

(FIG. 2D) Effect of glutaraldehyde (GTA) crosslinking on two OPN molecules. Many crosslinks between many amino acids contribute to extensive crosslinking and conformational and mobility changes.

FIGS. 3A to 3C provide graphical representation of the mechanical device used to characterize the OPN formulations.

(FIG. 3A) Rigid double-cantilever beam (RDCB) sample with dimensions.

(FIG. 3B) Depiction of RDCB sample during test.

(FIG. 3C) Typical load-opening curve and its associated dissipated energy.

FIGS. 4A to 4F provide the force-opening curves for the six tests performed on hydroxyapatite beams. Results are shown as the force applied (in Newtons) as a function of the opening (in μm).

(FIG. 4A) Results obtained using pure OPN on a bare hydroxyapatite substrate.

(FIG. 4B) Results obtained using OPN crosslinked with TG2 on a bare hydroxyapatite substrate.

(FIG. 4C) Results obtained using OPN crosslinked with TG2 on an OPN-coated hydroxyapatite substrate.

(FIG. 4D) Results obtained using OPN crosslinked with TG2 on an OPN/NC9 coated hydroxyapatite substrate.

(FIG. 4E) Results obtained using OPN crosslinked with GTA on a bare hydroxyapatite substrate.

(FIG. 4F) Results obtained using OPN crosslinked with GTA on an OPN-coated hydroxyapatite substrate.

FIG. 5 provides the force-opening curves for the tests Ref-dent, GTA-dent, GTA-C-dent performed on dentin beams. Results are shown as the force applied (in Newtons) as a function of the opening (in mm).

FIG. 6 provides the toughness values for the nine different adhesive-substrate groups of the study, compared contextually to the toughness of a widely used common adhesive (tape on glass). Results are shown as the fracture toughness (J/m²) as a function of the OPN formulation tested.

### DETAILED DESCRIPTION

The present disclosure provides using crosslinked OPN to treat an exposed surface of a mineralized tissue (e.g., bone, tooth, cartilage, etc.). In the Example below, it was determined that the cohesive/adhesive binding properties of OPN were increased by the covalent intermolecular crosslinks generated by crosslinking agents to enhance the fracture toughness of simulated bone. These results indicate that crosslinked OPN can thus be used to treat an exposed surface of a mineralized tissue, either to prevent or limit crack propagation or to adhere the exposed surface of the mineralized tissue to another surface (from another mineralized tissue or from a graft for example). In some embodiments, the mineralized tissue adhesive or filler composition of the present disclosure can be used to regulate the mineralization of the exposed surface of the mineralized tissue.

Without wishing to be bound to theory, it is understood that using crosslinked OPN is advantageous because it provides ductile properties (e.g., toughness) to mineral interfaces allowing resistance to mechanical loading (for example, in tension, bending and torsion) due to its crosslinking and the presence of sacrificial bonds. OPN also exhibits a high density of binding sites (functional groups) per molecule for mineral and calcium binding, and for crosslinking.

Many enzymes are known to modify components of extracellular matrices to modify their properties and/or to modulate resident cell behavior. For example, collagen is physiologically crosslinked through the action of the enzyme lysyl oxidase that catalyzes the formation of the lysine-derived aldehyde, allysine [19]. This process provides particularly bones, tendons and ligaments with high tensile strength [20]. The crosslinking of noncollagenous proteins such as OPN is predominantly conferred by the transglutaminase family of crosslinking enzymes, mainly tissue transglutaminase 2 (TG2) and Factor XIIIA. As shown in FIG. 2A, OPN includes glutamine and lysine residues in its primary structure—two amino acids that can be physiologically crosslinked by TG2 and Factor XIIIA, by forming an isopeptide bond [21, 22] (FIG. 2A, B). Kaartinen et al. [21] described that TG2 crosslinks two other substrates in addition to OPN—bone sialoprotein, and fetuin-A (α2HS-glycoprotein). Importantly, it has been previously demonstrated that TG2 increases the binding properties of OPN to collagen [23], and the number of TG2-crosslinked OPN complexes correlates with an increase in mechanical strain [24]. Finally, in cartilage lesions, application of TG2 provided an adhesive performance superior to that of fibrin [25].

Glutaraldehyde (GTA) is a well-known, extensively used industrial and clinical crosslinker (clinically, as part of GLUMA, see below) that has also been used for decades to preserve biological structure by “fixation” (i.e. crosslinking of organic material, mainly proteins, in biological tissues) of samples prepared primarily for microscopy and other protein immobilization/stabilization purposes [26]. GTA, as a dialdehyde-containing chemical, crosslinks proteins with a higher efficiency than the biological enzyme TG2 [27] (FIG. 2D), and it can be used as a reference to establish the degree of crosslinking of OPN in terms of its relative adhesive performance when compared to biological agents. Therefore, a person skilled in the art would consider crosslinking OPN with any physiologically acceptable form of GTA for its enhanced crosslinking ability. In some embodiments, GTA can be provided in a commercial, clinically approved form, such as in the GLUMA® universal bonding compound.

Systems for improving adhesion in teeth are known in the art and are usually composed of three fundamental steps aimed at providing micromechanical retention (a canonical three-step method; etching, priming and bonding). The first step seeks to prepare the substrate, e.g. tooth enamel, dentin, or cementum, by optimizing its surface for primer binding and permeation. This can be accomplished by acid treatment (e.g. phosphoric acid for example), and dissolution of mineral crystals (in teeth, the mineral is a form of calcium-phosphate [apatite] mineral) and is often termed “etching”. Etching provides a roughened exposed tooth surface that improves the micromechanical bonding that results initially from primer binding, and then from adding subsequent applied layers. After etching and rinsing, a second step involves application of a transient primer which renders the etched surface compatible with (wetted by) monomer bonding agent. Application of this monomer bonding agent is the third step of the sequence which provides micromechanical interlocking between the natural substrate and the subsequent dental restoration material. This third-step bonding compound usually comprises resins made up of methacrylates (e.g. 2,2-bis[4-(3-methacryloyloxy-2-hydroxypropoxy)phenyl]propane (bis-GMA), triethyleneglycol dimethacrylate (TEGDMA), and urethane dimethacrylate) which are crosslinked to increase adhesive strength. This bonding compound composition is also contained in the final restoration material which also incorporates fillers, coloring agents and other additives. Thus, these bonding systems rely solely on micromechanical adhesion to the mineralized tissue substrate. The purpose of these bonding systems is to retain the final restoration in place. Different generations of bonding systems have emanated from this fundamental three-step method. Other procedures use fewer steps, but don't change the nature of the micromechanical adhesion. One includes combining the primer and the bonding monomer into a single compound (two-step procedure). Another strategy is to combine etching and priming in one step, followed by the application of the bonding agent (two step no rinse procedure). Less frequently, all three steps (etching, priming and bonding) are combined into one step (thus a one-step no rinse procedure). In one embodiment, OPN is used as a natural chemical bonding agent as opposed to the micromechanical bonding system described above, within an etch-and-rinse three-step or two-step method. In another embodiment, OPN is used, in lieu or in combination with a resin, within a no-rinse two-step or one-step method.

In some embodiments, OPN provides augmented micromechanical bonding by virtue of its chemical affinity to mineral (chemical bonding). The term “chemical bonding” refers to ionic interactions which include, but are not limited to, ionic interactions between collagen type I and/or other noncollagenous proteins (such as osteocalcin and others) and the amino acid residue side chains in OPN, complexation of OPN with calcium ions and lattice (or amorphous) calcium in mineral or mineral precursors, interaction between OPN and polar functional groups (e.g. phosphate- or hydroxyl-). In this embodiment OPN provides adhesion between two surfaces (one of which is an exposed surface of a mineralized tissue) via micromechanical and chemical bonding. In such an embodiment, OPN can provide interfacial adhesion between the exposed surface of a mineralized tissue and another exposed surface of a mineralized tissue or a dental restoration as indicated below.

### Methods of Treating an Exposed Mineralized Tissue Surface

The present disclosure provides for using crosslinked OPN protein as a mineral tissue adhesive or filler composition capable of treating an exposed surface of a mineralized tissue. As used in the context of the present disclosure, an “exposed surface” of a mineralized tissue refers to a surface of a mineralized tissue which has lost or is susceptible of losing its integrity therefore exposing underlying cells or tissue which were not previously exposed to the extracellular milieu. In an embodiment, the exposed surface can be a naturally occurring exposed surface such as a crack (in the nano-, micro- or millimeter range) or a fracture. The term “fracture” includes, but is not limited to, greenstick and spiral fractures. In another embodiment, the exposed surface can be a chemically-created exposed surface, e.g. an exposed surface created by a chemical process. In another embodiment, the exposed surface can be a surgically-created exposed surface, e.g. an exposed surface created by a surgical procedure or a surgical instrument.

In an embodiment, the mineralized tissue adhesive/filler composition of the present disclosure can be used to increase “interface toughness” or “adhesive toughness”. These terms which are collectively used to refer to an amount of energy required to separate a surface (e.g., the surface of an exposed mineral tissue) from an adhesive (e.g., a mineralized tissue adhesive or filler).

As used in the context of the present disclosure, the expression “to treat” or “the treatment” of an exposed surface of a mineralized tissue refers to the ability of the mineralized tissue adhesive or filler composition to limit or prevent the lost in integrity of the expose surface and/or to restore, at least in part, the integrity of the exposed surface. For example, the mineralized tissue adhesive or filler composition can be used prophylactically to limit, reduce or retard further exposure of the mineralized tissue, such as, for example, by reducing or retarding crack propagation and/or bone fracture. This can be done by applying a coating of the mineralized tissue adhesive or filler on a mineralized tissue which is susceptible of cracking or fracturing. The mineralized tissue adhesive or filler composition can also be used therapeutically to restore the integrity of the exposure surface for example to fill a crack or glue a graft or a detached mineralized tissue fragment to the existing exposed mineralized tissue.

In some embodiments, the mineralized tissue adhesive or filler can be used to attach, permanently or temporarily, the exposed surface with another surface which can be an artificial/synthetic one such as a dental restoration. This can be done by applying a coating of the mineralized tissue adhesive or filler on a mineralized tissue which is has been determined to receive the dental restoration.

In an embodiment, the mineralized tissue is a tooth and the mineralized tissue adhesive or filler composition is applied to an enamel, a dentin or a cementum. In an embodiment, the mineralized tissue is a bone, such as, for example, a cortical bone.

Osteopontin (OPN) is a noncollagenous matrix protein that is found naturally in bones and teeth. OPN is a secreted protein that binds to hydroxyapatite with high affinity, and thus it regulates mineral crystal growth in the bone matrix and at interfaces (and similarly in teeth). In the context of the present disclosure, OPN can be obtained from a mammal, such as a human (and have, for example, Gene ID: 6696). In some other embodiments, OPN can be obtained from other nonhuman mammals. OPN can be purified from existing organisms, particularly from cow's milk, or can be produced recombinantly. OPN variants and/or fragments can also be used provided that such variants and fragments can be crosslinked by a crosslinking agent which crosslinks the wild-type OPN.

A variant comprises at least one amino acid difference when compared to the amino acid sequence of the native/known protein. As used herein, a variant refers to alterations in the amino acid sequence that do not adversely affect the biological functions of osteopontin (OPN). A substitution, insertion or deletion is said to adversely affect the protein when the altered sequence prevents or disrupts a biological function associated with OPN. For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of OPN. The protein variants have at least 50%, 55%, 60%, 65%, 70%, 75%, 80%, 85%, 90%, 95%, 96%, 97%, 98% or 99% identity to the wild-type OPN. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant osteopontin (OPN) described herein may be (i) one in which one or more of the amino acid residues are substituted with a conserved or nonconserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. A “variant” OPN can be a conservative variant or an allelic variant.

The osteopontin (OPN) can be a fragment of a known/native protein or fragment of a variant of OPN. In an embodiment, the fragment corresponds to the known/native OPN protein to which the signal peptide sequence has been removed. In some embodiments, OPN protein “fragments” have at least at least 100, 125, 150, 175, 200, 225, 250, 275 or 300 or more consecutive amino acids of the wild-type OPN. A fragment comprises at least one less amino acid residue when compared to the amino acid sequence of the known/native OPN and still possess the biological activity of the full-length OPN. In an embodiment, the fragment corresponds to the amino acid sequence of the OPN protein lacking the signal peptide. In some embodiments, fragments of the OPN protein can be employed for producing the corresponding full-length OPN by peptide synthesis. Therefore, the fragments can be employed as intermediates for producing the full-length OPN protein.

In an embodiment, the mineralized tissue adhesive or filler composition of the present disclosure is contacted/applied (directly or indirectly) to the exposed surface of a mineralized tissue which may include cracks, porosities or holes (microscopic or macroscopic), fractured surfaces or surgically created surfaces (as per sawing or drilling or cutting by any other means). The exposed surface of the mineralized tissue can include mineralized collagen and generally mineralized extracellular matrix. The exposed surface of the mineralized tissue can be at least in part calcified. As such, the mineralized tissue adhesive or filler composition can be contacted/applied directly or indirectly onto a calcified exposed surface. The exposed surface can include hydroxyapatite. As such, the mineralized tissue adhesive or filler composition can be contacted/applied directly or indirectly onto an exposed surface comprising mineralized collagen (and noncollagenous proteins) and hydroxyapatite (or other ion-substituted apatite phases). When the mineralized tissue is a tooth, the mineralized tissue adhesive/filler composition of the present disclosure can be contacted/applied directly or indirectly onto the enamel and/or the dentin and/or the cementum as well as surfaces bridging enamel and dentin, enamel and cementum and/or dentin and cementum.

In another embodiment, the mineralized tissue adhesive or filler composition of the present disclosure is contacted/applied (directly or indirectly) to an exposed natural surface of a mineralized tissue. The term “exposed natural surface” may include cracks, porosities or holes (microscopic or macroscopic), fractured surfaces or surgically created surfaces (as per sawing or drilling or cutting by any other means). The exposed natural surface of the mineralized tissue can include mineralized collagen and generally mineralized extracellular matrix. The exposed natural surface of the mineralized tissue can be at least in part calcified. As such, the mineralized tissue adhesive or filler composition can be contacted/applied directly or indirectly onto a calcified exposed natural surface, with or without etching. The exposed natural surface can include hydroxyapatite (or other ion-substituted apatite phases). As such, the mineralized tissue adhesive or filler composition can be contacted/applied directly or indirectly onto an exposed natural surface comprising mineralized collagen (and noncollagenous proteins) and hydroxyapatite (or other ion-substituted apatite phases). When the mineralized tissue is a tooth, the mineralized tissue adhesive/filler composition of the present disclosure can be contacted/applied directly or indirectly onto the exposed natural surfaces which may include enamel and/or dentin and/or cementum, including their interfaces.

In another embodiment, the mineralized tissue adhesive or filler composition of the present disclosure is contacted/applied (directly or indirectly) to an artificial (synthetic) surface. The term “artificial surface” includes surfaces of any restorative material retained by an adhesive and includes, but is not limited to, surfaces on bridges, cements, implants, crowns, caps, veneers, sealants, fillings and decorative attachments, and orthodontic brackets, bands, hooks, buttons and any other fixtures. The mineralized tissue adhesive or filler composition can also be contacted/applied directly or indirectly onto a calcified artificial surface. The artificial surface can include mineralized collagen (and noncollagenous proteins) and hydroxyapatite (or other ion-substituted apatite phases). As such, the mineralized tissue adhesive or filler composition can be contacted/applied directly or indirectly onto an artificial surface comprising mineralized collagen and hydroxyapatite.

In one embodiment, the mineralized tissue adhesive or filler composition of the present disclosure is contacted/applied (directly or indirectly) between at least two exposed natural surfaces. In another embodiment, the mineralized tissue adhesive or filler composition of the present disclosure is contacted/applied (directly or indirectly) between at least two artificial surfaces. In another embodiment, In one embodiment, the mineralized tissue adhesive or filler composition of the present disclosure is contacted/applied (directly or indirectly) between at least one exposed natural surface and at least one artificial surface.

When the mineralized tissue is a tooth, the at least one artificial surface can be contacted/applied with the adhesive or filler composition of the present disclosure onto the enamel and/or dentin and/or cementum as well as surfaces bridging enamel and dentin, enamel and cementum and/or dentin and cementum and their interfaces.

When the mineralized tissue is a bone, the at least one exposed natural surface can be contacted/applied with the adhesive or filler composition of the present disclosure onto an osseointegrated implant. The term “osseointegrated implant” including, but not limited to, plates, rods, screws, and/or wires.

As indicated in the Example, in some embodiments, it may be advantageous to deposit a layer of monodisperse osteopontin (OPN) protein between the mineralized tissue adhesive or filler composition and the exposed surface of the mineralized tissue to increase adherence between the exposed surface and the crosslinked OPN protein. As used in the present disclosure, the terms “monodisperse” refer to OPN proteins being in a monomeric form, e.g. lacking intermolecular covalent association between OPN proteins.

The exposed surface of the mineralized tissue includes mineralized collagen and mineralized extracellular matrix, and in some embodiments, a layer of monodisperse osteopontin (OPN) protein is first directly or indirectly deposited onto a surface of mineralized tissue that can comprise mineralized collagen. Without wishing to be bound to theory, it is understood that the monodisperse form of OPN protein adhere well to the exposed surface of the mineralized tissue and increases the adherence of the crosslinked OPN protein to the exposed surface of the mineralized tissue. The mineralized tissue adhesive or filler composition of the present disclosure can be contacted/applied directly onto the layer of monodisperse OPN protein that has been deposited on the exposed surface of mineralized collagen. It is expected that the contact between the crosslinked OPN protein and the layer of monodisperse OPN protein will cause a polymerization of the monodisperse OPN protein, at least at the interface between the crosslinked OPN protein and the layer of monodispersed OPN protein, and in some embodiments, all the way down to the mineralized tissue exposed surface. As such, even though a layer of monodisperse OPN protein is directly deposited on the exposed surface, after the crosslinked OPN protein is contacted with the layer of monodisperse OPN protein, some of the monodisperse OPN protein will be crosslinked (at least at the interface with the crosslinked OPN protein, thus providing a continuum of crosslinked OPN across the defect). In some embodiments, the majority or the totality of the monodisperse OPN protein of the layer will be crosslinked after the contact with the crosslinked OPN protein.

The monodisperse osteopontin (OPN) protein can be provided in a solid dehydrated form (e.g., lyophilized for reconstitution) or can be provided in a liquid (hydrated) form.

The exposed surface of the mineralized tissue can be calcified. As such, a layer of monodisperse osteopontin (OPN) protein can be directly or indirectly deposited onto an exposed surface of a calcified mineralized tissue. The mineralized tissue adhesive or filler composition of the present disclosure can be contacted/applied directly or indirectly onto the layer of monodisperse OPN protein that has been deposited on the calcified surface of the mineralized tissue.

The surface of the mineralized tissue can include hydroxyapatite. As such, a layer of monodisperse osteopontin (OPN) protein can be directly or indirectly deposited onto a surface of mineral tissue comprising hydroxyapatite (or other ion-substituted apatite phases). The mineralized tissue adhesive or filler composition of the present disclosure can be contacted/applied directly or indirectly onto the layer of monodisperse OPN protein that has been deposited on the mineralized tissue surface comprising hydroxyapatite.

When the surface is a tooth, a layer of monodisperse osteopontin (OPN) protein can be applied directly or indirectly on the enamel and/or the dentin and/or the cementum including a surface bridging, the enamel and the dentin, the enamel and the cementum and/or the dentin and the cementum. The mineralized tissue adhesive/filler composition of the present disclosure can be contacted/applied directly or indirectly onto the layer of monodisperse OPN protein that has been deposited on the enamel, dentin and/or cementum.

The mineralized tissue adhesive or filler composition of the present disclosure can be provided in a solid dehydrated form (e.g., lyophilized for reconstitution) or can be provided in a liquid (hydrated) form. The mineralized tissue adhesive or filler composition of the present disclosure comprises intermolecular crosslinked osteopontin (OPN) protein. The expression “intermolecular crosslinked OPN protein” refers to the fact that the mineralized tissue adhesive or filler of the present disclosure includes OPN protein which are associated, via covalent bonds, with other OPN protein and may be considered to be polymers or in polymeric form. The covalent bonds between two OPN protein of the mineralized tissue adhesive or filler can be isopeptide bonds, any other type of covalent linkage or a mixture of isopeptide bonds with other bonds. In an embodiment, the isopeptide bonds between two OPN protein molecules can involve a glutamine residue on a first OPN protein and a lysine residue on another OPN protein, as illustrated in FIG. 2B. In the mineralized tissue adhesive or filler composition of the present disclosure, there may be one or more bonds between two OPN proteins. Furthermore, a single OPN protein can be covalently associated with one or more additional OPN proteins. In some embodiments, the crosslinked OPN protein can also include intramolecular crosslinking, e.g., the presence of covalent bonds (excluding the amino bond) between amino acid residues of the same OPN protein. The mineralized tissue adhesive or filler composition of the present disclosure can include additional components, including, but not limited to, a buffer, a mineral, etc.

The mineralized tissue adhesive or filler composition of the present disclosure includes crosslinked osteopontin (OPN) protein and as such, the methods of the present disclosure can also include providing and/or preparing the mineralized tissue adhesive or filler composition. In some embodiments, the mineralized tissue adhesive or filler composition can be prepared by contacting a monodisperse OPN protein composition (which, in some embodiments in which the monodisperse OPN protein composition is provided in a solid dried form, has been reconstituted as a liquid) with a crosslinking agent under conditions (time, temperature, concentrations, etc.) allowing the crosslinking of the OPN protein. The person of ordinary skills in the art would know how to adapt the crosslinking conditions, which depend, amongst other things, on the type of crosslinking agent used and the geometry of the exposed surface to achieve sufficient crosslinking of the OPN protein. In some embodiments, the crosslinking agent can be an enzyme (for example in a purified form) known to be able to crosslink OPN protein. Enzymes capable of crosslinking OPN protein, include, but are not limited to transglutaminases or TG (such as transglutaminase 2 or TG2) and/or Factor XIIIA. In an embodiment, when the crosslinking agent is TG2, the weight ratio used for the crosslinking step between TG2 and OPN is between about 1:2 to about 1:400. In some specific embodiments, the enzyme capable of crosslinking OPN protein and used to make the mineralized tissue adhesive or filler composition is TG2. In another embodiment, the crosslinking agent can be a chemical entity capable of crosslinking OPN protein. Chemical entities capable of crosslinking OPN protein include, but are not limited to, glutaraldehyde (or GA), genipin as well as 1-ethyl-3-(3-dimethylaminopropyl)carbodiimide hydrochloride (EDC). In an embodiment, when the crosslinking agent is GA, the weight ratio used for the crosslinking step between GA and OPN is between about 2:1 to about 1:10.

The mineralized tissue adhesive or filler composition can be contacted with, optionally in combination with a layer of monodisperse osteopontin (OPN) protein, an exposed surface of a mineralized tissue in a subject (such as, for example, a human subject or a non-human animal, such as a mammal). Alternatively or in combination, the mineralized tissue adhesive or filler composition can be included, optionally in combination with the layer of monodisperse OPN protein, in a medical device comprising a mineralized tissue or a mineralized tissue-derived material or intended to be used in contact with a mineralized tissue, such as a bone, a tooth or a cartilage (such as a graft).

### Kit for Using the Mineralized Tissue Adhesive or Filler

The mineralized tissue adhesive or filler composition of the present disclosure can be used to treat an exposed surface of a mineralized tissue. The mineralized tissue adhesive or filler composition can be used to prevent or limit the lost in integrity of the mineralized tissue or bring together the surface of a mineralized tissue with the surface of another material (another mineralized tissue or a graft for example). Since the mineralized tissue adhesive or filler composition of the present disclosure can be made rapidly and should preferably be made within minutes of applying to the exposed surface of the mineralized tissue, the present disclosure also provides a kit for using (and in some embodiments making) the mineralized tissue adhesive or filler composition. The kit comprises a monodisperse osteopontin (OPN) composition, a crosslinking agent as well as instructions on how to use the mineralized tissue adhesive or filler composition. The instructions can include, without limitations, how to prepare a mineralized tissue adhesive or filler composition from the monodisperse OPN protein composition, optionally how to prepare a monodisperse solution of OPN protein from the monodisperse OPN protein composition, optionally how to deposit a layer of monodisperse OPN protein on an exposed surface of a mineralized tissue to form a layer of monodisperse OPN protein as well as how to contact the mineralized tissue adhesive or filler composition on the exposed surface of the mineralized tissue or on the layer of monodisperse OPN protein.

The kit first comprises a monodisperse osteopontin (OPN) protein composition. The monodisperse OPN protein composition is intended to be used to make the mineralized tissue adhesive or filler and, and in some embodiments, to also generate the layer of monodisperse OPN protein. The monodisperse OPN protein composition can be provided in a solid, dehydrated form. In such embodiments, the instructions can include how to rehydrate the monodisperse OPN protein composition with an aqueous solution (such as water, a saline or a buffer) and the kit can optionally include such solvent for reconstitution. The monodisperse OPN protein composition can be provided in a liquid form. In the kit, the instructions can indicate that at least one fraction (or the entirety) of the monodisperse OPN protein composition is crosslinked by contacting the OPN protein with any of the crosslinking agents listed above under conditions (time, temperature, concentrations, etc.) allowing the crosslinking of the OPN protein. The instructions of the kit can also indicate that the mineralized tissue adhesive or filler composition is to be applied directly on the exposed surface of the mineralized tissue.

The kit also comprises a crosslinking agent capable of crosslinking of the osteopontin (OPN) protein. The crosslinking agent can be provided in a distinct container than the one used for the monodisperse OPN protein composition. In some embodiments, the crosslinking agent can be an enzyme (for example in a purified form) known to be able to crosslink OPN protein. Enzymes capable of crosslinking OPN protein, include, but are not limited to transglutaminases or TG (such as TG2) and/or Factor XIIIA. In some specific embodiments, the enzyme capable of crosslinking OPN protein and used to make the mineralized tissue adhesive or filler is TG2. In an embodiment, when the crosslinking agent is TG2, the weight ratio used for the crosslinking step between TG2 and OPN is between about 1:2 to about 1:400. In another embodiment, the crosslinking agent can be a chemical entity capable of crosslinking OPN protein. Chemical entities capable of crosslinking OPN protein include, but are not limited to, glutaraldehyde (or GA), genipin as well as 1-ethyl-3-(3-dimethylaminopropyl)carbodiimide hydrochloride (EDC). In an embodiment, when the crosslinking agent is GA, the weight ratio used for the crosslinking step between GA and OPN is between about 2:1 to about 1:10.

In some embodiments, in the kit, the instructions can also indicate that another fraction of the monodisperse osteopontin (OPN) composition is not submitted to a crosslinking step and is instead used to form a layer of monodisperse OPN protein on the exposed surface of the mineralized tissue. In such embodiments, the monodisperse OPN composition can be reconstituted from a solid form, diluted from a concentration solution or be in a “ready-to-use” form. The instructions of the kit can also indicate that the layer of monodisperse OPN protein is to be applied directly on the exposed surface and that the mineralized tissue adhesive or filler is to be applied directly on the layer of monodisperse OPN protein.

The instructions of the kit can include how to use the mineralized tissue adhesive or filler on an exposed surface of a mineralized tissue. For example, the instructions can include contacting/applying the mineralized tissue adhesive or filler composition of the present disclosure (directly or indirectly via a layer of monomeric osteopontin [OPN] protein) to the an exposed surface which may include cracks, porosities or holes (microscopic or macroscopic). The surface of the mineralized tissue can include mineralized collagen and, in some embodiments, the mineralized tissue adhesive or filler composition of the present disclosure can be contacted/applied (directly or indirectly via a layer of monomeric OPN protein) onto a surface of mineralized collagen. The exposed surface of the mineralized tissue can be calcified. As such, the mineralized tissue adhesive or filler composition can be contacted/applied (directly or indirectly via a layer of monomeric OPN protein) onto a calcified mineral tissue surface. The surface of the mineralized tissue can include hydroxyapatite. As such, the mineralized tissue adhesive or filler composition can be contacted/applied (directly or indirectly via a layer of monomeric OPN protein) onto an exposed surface comprising mineralized collagen and hydroxyapatite. When the surface is a tooth, the mineralized tissue adhesive or filler composition of the present disclosure can be contacted/applied (directly or indirectly via a layer of monomeric OPN protein) on the enamel and/or the dentin and/or the cementum as well as a surface bridging enamel and dentin, enamel and cementum and/or dentin and cementum.

The kit of the present disclosure can include one or more applicators to deposit the layer of monodisperse osteopontin (OPN) protein and/or the mineralized tissue adhesive or filler. In an embodiments, the same applicator can be used to successively deposit the layer of monodisperse OPN protein and the mineralized tissue adhesive or filler composition on the exposed surface of the mineralized tissue.

The kit of the present disclosure can also include a container to mix and combine the monodisperse osteopontin (OPN) protein composition and the crosslinking agent.

The kit can provide distinct containers for providing the monodisperse osteopontin (OPN) protein composition and the crosslinking agent. In some embodiment, the kit can provide an ampoule with at least two distinct container sections separated by a membrane. In such embodiment, the membrane of the ampoule is designed to be detachable, tearable or breakable so as to allow the contact between the monodisperse OPN protein composition and the crosslinking agent. In such embodiment, the kit can also provide a further container for providing the monodisperse OPN protein intended to be deposited on the exposed surface so as to form a layer of monodisperse OPN protein on the mineralized tissue.

### Dental Applications of the Adhesive

The mineralized tissue adhesive or filler composition of the present disclosure is especially useful in dental applications to limit the degradation or preserve the integrity of an exposed surface of a tooth. The mineralized tissue adhesive or filler composition of the present disclosure can be used (alone or in combination, simultaneously or sequentially, with other components such as, for example, a layer of monodisperse osteopontin (OPN) protein and/or a mineralization solution of calcium and/or a mineralization solution of phosphate) as a dental protective coating. In such embodiment, the mineralized tissue adhesive or filler composition can be applied to a tooth having or susceptible of having nanostructural/microstructural/macrostructural defects (cracks, cavities, etc.) to prevent or limit crack aggravation, tooth fracture and/or cavities. In some embodiments, the mineralized tissue adhesive or filler can be used to regulate mineralization of an exposed surface of a tooth. In some specific embodiments, the mineralized tissue adhesive or filler composition can be used to limit, retard or treat an incipient carious lesion. The mineralized tissue adhesive or filler composition of the present disclosure can be used (alone or in combination, simultaneously or sequentially, with other components such as, for example, a layer of monodisperse OPN protein and/or a mineralization solution of calcium and/or a mineralization solution of phosphate) as a dental filling. In such embodiment, the mineralized tissue adhesive or filler composition can be applied to a crack or a cavity to strengthen the tooth. The mineralized tissue adhesive or filler composition of the present disclosure can be used (alone or in combination, simultaneously or sequentially, with other components such as, for example, a layer of monodisperse OPN protein and/or a mineralization solution of calcium and/or a mineralization solution of phosphate) as a dental bonding or veneer. In such embodiment, the mineralized tissue adhesive or filler composition can be applied to a bonding or veneer to increase its strength.

The present invention will be more readily understood by referring to the following examples which are given to illustrate the invention rather than to limit its scope.

### EXAMPLE

### Materials and Methods

Proteins, enzymes and reagents. Purified, well-characterized bovine milk-derived osteopontin (OPN) provided Dr. Esben Sørensen and Arla Foods was used in this study (University of Aarhus, Denmark, and Arla Foods, Viby, Denmark) [14, 30, 31]. Briefly, the extraction of OPN consisted of a two-step protocol where the proteose peptone was obtained from milk, from which OPN was extracted. Centrifugation, heating and cooling, and pH adjustment separated caseins and denatured whey proteins from solution. Trichloroacetic acid-precipitated proteins, including OPN, were purified using urea and Sephadex G-75 gel chromatography.

Two crosslinking agents (one physiological, an enzyme found in bone, and the other a small-molecule chemical crosslinker) were used to model a toughness mechanism for OPN interactions in bone. The first crosslinker used was the enzyme tissue transglutaminase 2 (TG2) which is abundant in bone, as extracted and purified from guinea pig liver (Sigma Aldrich Cat# T5398). As a control, to specifically inhibit TG2 activity, an irreversible inhibitor of TG2 known as NC9 was used; this small molecule occupies a surface site on TG2 with the direct result of forcing the enzyme into an open conformation that inhibits its activity [32]. NC9 inhibitor was used in this study in combination with TG2 to verify that the increase in toughness was indeed correlated with the enzymatic action of TG2. The effect of the small-molecule, chemical fixative glutaraldehyde was also examined (GTA, Electron Microscopy Sciences, Cat#16210) on OPN toughness, since GTA crosslinks collagens and noncollagenous proteins with a higher efficiency than does the physiologic enzyme TG2 [27]. Calcium chloride, Tris-HCl buffer and all other reagents were from Sigma unless otherwise specified.

Sample preparation. A well-controlled, interface-substrate mechanical testing system was used (see FIG. 3A) to measure the adhesion produced by OPN when administered between apposing substrate synthetic hydroxyapatite and biogenic tooth dentin slabs/beams. With a thin layer of aqueous OPN solution sandwiched between two hydroxyapatite beams pressed into contact, a miniaturized mechanical testing device was used to fracture the interface and measure the energy consumed in the process (toughness). The different OPN preparations (see below and Table 1) were used on two types of flat substrates: synthetic hydroxyapatite and biogenic tooth dentin. A commercially available hydroxyapatite was used (Himed Old Bethpage, N.Y., USA) for the synthetic mineral model. For the biogenic substrate, cortical bovine bone was at first considered and tried, but the inherent porosity (e.g. vascular and remodeling canals) and heterogeneities of bone structure [33] made it difficult to produce consistent, comparable beams. Tooth dentin is avascular and relatively homogeneous, and is a well-accepted alternative for in vitro bone cell-matrix/mineral interaction studies, as it shares a roughly similar mineral, collagen and noncollagenous proteins content as bone. More specifically, a narwhal-tusk dentin was used (Department of Fisheries and Ocean, Winnipeg) because of its particular homogeneity, low porosity and large dimensions necessary for us to prepare cantilever substrate beams for our testing apparatus. Another advantage for selecting dentin was that the polished dentin and synthetic hydroxyapatite surfaces had a similar surface roughness (data not shown).

TABLE 1
Summary of the formulations tested in this example

| Group | Adhesive | Substrate |
|---|---|---|
| (Ref) | Pure OPN | Bare HA surface |
| (TG) | OPN crosslinked with TG2 | Bare HA surface |
| (TG-C) | OPN crosslinked with TG2 | OPN-coated HA surface |
| (TG-C-NC9) | OPN crosslinked with TG2 | OPN-coated HA surface |
| | and NC9 | |
| (GTA) | OPN crosslinked with GTA | Bare HA surface |
| (GTA-C) | OPN crosslinked with GTA | OPN-coated HA surface |
| (Ref-dent) | Pure OPN | Bare dentin surface |
| (GTA-dent) | OPN crosslinked with GTA | Bare dentin surface |
| (GTA-C-dent) | OPN crosslinked with GTA | OPN-coated dentin surface |

Pairs of rectangular beams (dimensions 8×2×1 mm) were cut from both mineral substrates using a diamond saw (Accutom-5, Struers, Denmark). The beams, which served as substrates for the OPN adhesive formulations, were then attached onto custom-made steel fixtures using epoxy (FIG. 3A). The steel fixtures were longer than the substrates, and included pinholes which were used to transmit opening forces to the sample (FIG. 3A) in a configuration similar to a rigid double-cantilever beam (RDCB) [34-36]. The open faces of the substrates were then polished using metallographic methods on a polishing wheel down to a particle size of 0.05 μm. The two pairs of RDCB fixtures were stored in deionized water at room temperature until the preparation of the adhesive and mechanical testing, all of which occurred within four hours (including the incubation time for the OPN solutions, see below).

To prepare these samples for incubation with OPN solutions, the RDCB mineral beams were first washed with acetone, and then with ethanol. They were then extensively rinsed with deionized water and dried under air flow at room temperature. The basic protein incubation procedure consisted of placing 2.5 μL of 100 g/L (2.96 mM) OPN solution onto each of the two RDCB mineral substrates (corresponding to 250 μg of OPN for each substrate or unit of surface, for a combined total of 500 μg of OPN in the assembled interface). Differently treated substrate beams (for details of formulations and crosslinking see below) were then apposed and loaded with a 60 g weight, and together transferred into a sealed box that contained a small reservoir of water to maintain 100% humidity, and the sealed box was then placed into an incubator at 37° C. (physiologic temperature) for two hours for proper crosslinking of OPN [37]. The box was then removed from the incubator and the samples were immediately tested using a miniaturized mechanical testing machine as detailed below.

As indicated in Table 1, there were multiple variations and additions for crosslinking based on the basic procedural plan described above.

A total of nine combinations of OPN-based adhesives and substrates were considered in this study. The preparation of the samples was based on a “reference” protocol which was used to create the sample group labeled (Ref). The reference protocol was a basic protocol where noncrosslinked OPN was deposited on the substrates.

For samples in the group labeled (TG), OPN was cross-linked into a polymerized network with the enzyme tissue transglutaminase (TG2). Transglutaminases are calcium-dependent enzymes, and require calcium-binding to catalyze a crosslinking reaction in OPN [38]. Crosslinked OPN was prepared by mixing 7.5 μL of 100 g/L (2.96 mM) of OPN solution, 5.25 μL of TG2 solution (2 mU/μL), and 2.25 μL Reaction Buffer (64 mM Tris-HCl, pH 8, 20 mM CaCl2, and 6.4 mM dithiothreitol) for a total volume of 15 μL. Tris-HCl was used to maintain pH at 8 during the chemical reaction between TG2 and OPN, and the addition of dithiothreitol (DTT) solution was used as a reducing agent to keep the active sites (thiol groups) of the enzyme open for chemical reaction [38]. Each mineral beam received 5 μL of the mixture before assembling them in apposition. These samples were then incubated for 2 h at 37° C.

Samples for the pre-coated group TG-C followed the same procedure, but the substrates were pre-coated with pure OPN. For that purpose, 5 μL of 100 g/L (2.96 mM) of the OPN solution was applied to the surface of each of the substrates, which were then left at room temperature for 2 h for drying before the adhesive was applied.

Samples from the group TG-C-NC9 were prepared using the same procedure as for TG-C, but a solution of 2.5 μL of a 6 mM NC9 solution was additionally added. The total volume of the mixture was then 17.5 μL, and each substrate received 5.83 μL of the mixture before assembling the beams together and incubation. The total quantity of OPN used was also 500 μg for these samples.

GTA is a universal protein crosslinker that we also used in this study; as an aliphatic dialdehyde, it crosslinks proteins rapidly, effectively and irreversibly [26]. Glutaraldehyde reacts with many nucleophiles, of which amine groups in proteins contribute the most to the crosslinking since they are in the greatest abundance. Sulfhydral groups from cysteine and imidazole side chains of histidine also participate in the crosslinking reaction within and between proteins. To obtain these samples we used the same basic procedure, but with 7.5 μL of a 5% GTA solution added to 7.5 μL of the OPN solution concentrated at 100 g/L (2.96 mM). Each of the beams received 5 μL of this mixture before apposition and incubation. The quantity of OPN between the two substrates was the same for the monomeric OPN samples and the GTA-crosslinked OPN samples (250 μg on each substrate or unit of surface, for a total of 500 μg). This protocol was used on bare hydroxyapatite beams (group GTA) and OPN pre-coated hydroxyapatite beams (group GTA-C).

Finally, three groups were prepared where natural narwhal dentin was used in place of the synthetic hydroxyapatite as the cantilever beams (Table 1). Pure OPN monomer on dentin (group Ref-dent), OPN crosslinked with GTA on dentin (group GTA-dent), and OPN crosslinked with GTA on OPN-coated dentin (group GTA-C-dent).

Mechanical testing protocol. A custom-made fixture supported by two pins, with each of the pins sliding into the pinholes of the RDCB sample beams (FIG. 3B). This setup was designed for the pins to transmit the opening force F onto the sample. The force was recorded with a 5 lb capacity load cell, while the opening was measured with a linear variable differential transformer (LVDT). During the test, the opening was slowly increased at a rate of 3 μm/s, which opened the beams and progressively separated the OPN interface until complete failure. The test was stopped once the force had dropped to values below 0.5 N. A typical force-opening curve is shown in FIG. 3C. From this data, the work-of-fracture of the OPN interface was computed, obtained by dividing the energy under the force-opening curve (unit of Joule) by the initial area of the bond (unit of m²). The work-of-fracture (in J/m²) provided a measure of the toughness of the OPN-based adhesives on hydroxyapatite and dentin, corresponding to the amount of energy required to separate a unit surface of that adhesive.

### Results and Discussion

FIG. 4 shows the force-opening curves for the first six groups of adhesives (OPN on synthetic hydroxyapatite beams). Pure monomeric OPN solutions (Ref group) on the bare hydroxyapatite beams showed no evidence of adhesion, which underscored the necessity of crosslinking the protein to generate adhesion [23]. Without wishing to be bound to theory, this is likely explained by the rapid engagement of the majority of the mineral-reactive side groups of OPN (carboxylates from Asp and Glu, and phosphates from P-Ser) [39] on one hydroxyapatite beam or the other prior to the weighted apposition step of the procedure. In all other subsequent groups/experiments, OPN was crosslinked into a polymer network, producing force-opening curves which increased essentially monotonically up to a maximum force, followed by a progressive decrease of force up to a maximum opening of about 200 μm at failure. The progressive failure of some samples suggests toughening mechanisms such as crack bridging by intact proteinaceous ligaments. The experimental approach of the present example to measuring adhesion is at a much larger scale and using different substrates than that used by Fantner et al. [6], who elegantly assessed pulling forces on OPN at the single-molecule scale using atomic force microscopy.

Crosslinking of OPN by the addition of TG2 produced a tougher adhesive. A pre-coating of OPN onto the hydroxyapatite beam further increased the toughness of the interface, to values slightly above 10 J/m². Pre-coating hydroxyapatite beams with OPN provided an initial surface composed of both hydroxyapatite and OPN, a combination that appeared to be more favorable to subsequent OPN binding. It is likely that the secondarily added OPN molecules in solution developed linkages with OPN molecules from the pre-coat that in turn were already strongly bound (as monomer) to the hydroxyapatite mineral of the beam, a two-step process which resulted in a higher overall toughness. Indeed, monomeric OPN binds strongly to HA [40, 41], and previous work demonstrated that OPN molecules interact together homotypically with each other, and that the strength of OPN-OPN bonds is large enough to influence self-assembly and adhesion on mineralized tissue [39].

The results also show that the TG2 inhibitor NC9 inhibited TG2 crosslinking, so that samples from the group TG-C-NC9 exhibited negligible adhesion, similar to that observed for pure monomeric OPN. As expected, the strong chemical crosslinker GTA increased the adhesive performance of OPN significantly more than the enzymatic crosslinking by TG2; toughness was 33% higher by crosslinking OPN with GTA compared to crosslinking with TG2 (noncoated substrates), and 124% higher using OPN-coated substrates. The highest toughness we measured was about 25 J/m².

As shown in Table 1, dentin was also used as substrate to measure the toughness of three OPN-based adhesives: pure OPN (Ref-dent group), OPN crosslinked with GTA (GTA-dent group) and OPN crosslinked with GTA on OPN-coated dentin (GTA-C-dent group). Representative force-opening curves for these three groups are shown in FIG. 5. Interestingly, pure monomeric OPN did in this case provide adhesion on dentin, a result that contrasted with the lack of adhesion for pure monomeric OPN on hydroxyapatite. Besides slight differences in the biogenic dentin compared to the synthetic pure hydroxyapatite, it is possible that the presence of organic dentin components such as collagen and/or other noncollagenous proteins provided additional binding of the applied OPN even though they were not crosslinked, an additional binding which was sufficient to detect a small, measurable adhesion between the substrate beams. The GTA-dent and GTA-C-dent groups produced toughness values which were significantly higher than the Ref-dent group.

A summary of all the results is shown in FIG. 6. To provide relative context using the same mechanical device and setup, the toughness values we obtained for OPN were compared to that of a well-known standard adhesive—“office tape” on smooth glass substrate beams [35]. In seven tests out of nine, the adhesive performance of OPN was significantly greater than that of tape on glass. OPN adhesion was 3 times (Ref-dent group) to 17 times (GTA-C-dent group) superior to tape adhesion.

Overall, the results show that crosslinking of OPN into a polymer network after application between two mineral slabs increases toughness when assessed by mechanical testing. Moreover, chemical treatment with GTA is a more efficient crosslinker than enzymatic treatment with TG2 enzyme, but both treatments indeed showed remarkable toughness gains compared to using monomeric OPN alone (without crosslinking). Pre-coating the mineral substrate beams with OPN significantly improved adhesion as toughness increased by 20-70% on the hydroxyapatite substrate, and by 240% on dentin substrate. The GTA-dent group did not exhibit significant changes in toughness when compared to the same test on hydroxyapatite (GTA, p>0.05). Of particular note, the overall adhesive performance of OPN on the organic-inorganic composite dentin was superior to OPN on hydroxyapatite mineral alone; the Ref-dent group exhibited a toughness of 6.3 J/m² while the Ref group exhibited no toughness, and the GTA-C-dent group was 62% higher than the GTA-C group. Superior adhesion using crosslinked OPN and biogenic dentin beams/slabs as compared to the hydroxyapatite beams likely results from the mineral phase of dentin having a preferred orientation relative to the synthetic sintered hydroxyapatite, and from the fact that TG2 crosslinking of OPN enhances binding to collagen (as present in the dentin sample) [23].

TG2 found in bone crosslinks proteins (including OPN) predominantly by forming γ-glutamyl-249 -lysine isopeptide bonds between the polypeptide chains of proteins, as shown schematically in FIGS. 2A, B [12]. This inter- and intramolecular crosslinking between glutamine and lysine leads to changes in protein conformation from the open flexible monomeric state, to the heavily crosslinked polymeric state [42] (FIG. 2B). FIG. 2C shows the chemical reaction leading to a γ-glutamyl-ε-lysine bond. Sorensen [12] demonstrated that this type of bond was predominant in OPN crosslinked by TG2 and occurred between Gln42 and Lys4, and Gln193 and Lys154, Lys157 or Lys231. In addition, Gln248 [43], Gln34 and Gln36 [14] are also three other TG2-reactive glutamines. In the case of Gln34 and Gln36, the sequence alignments containing these glutamines are conserved in all known OPN, thus suggesting a functional importance at these particular sites [14]. The very precise TG2-mediated crosslinking sites differ from the essentially indiscriminate, nonphysiologic crosslinking reaction of proteins with GTA where polymerization products mainly occur as a result of extensive crosslinking between many functional groups of amino acids such as amine, thiol, phenol, and imidazole groups. Using GTA, many other amino acids (lysine, tyrosine, phenylalanine, tryptophan, histidine, proline, serine and others) of the OPN amino acid sequence are expected to react with the aldehydes, forming many other linkages for adhesion as depicted in FIG. 2D [44]. The higher number of linkages in OPN molecules crosslinked nonphysiologically by the chemical GTA leads to higher changes in the conformation and adhesive function of OPN.

In summary, the present example provides evidence that polymer networks of OPN established by the crosslinking action of the enzyme TG2 may play an important functional adhesive/cohesive role in bone. In addition to being dispersed throughout the bone extracellular matrix, OPN is also enriched in the skeleton at the interface where new bone is deposited onto older bone in cement lines (actually planes in three dimensions) as part of the bone remodeling cycle. At both these locations, adhesion afforded by crosslinking would seem to be advantageous across different length scales—a feature particularly important given the highly hierarchical nature of bone structure. At the molecular scale, binding and crosslinking interactions of OPN between and with collagen fibrils and/or mineral crystallites likely participate in dissipating energy under mechanical strain, as do sacrificial bonds in the extracellular matrix. The present study has revealed that the monomeric form of OPN, when applied onto a pure hydroxyapatite substrate, cannot develop bonds sufficiently strong enough to be adhesive; in this case, the presence of TG2 appears to be a necessary requirement for an adhesive function. However, monomeric OPN binding to dentin does provide some adhesion, likely attributable to interactions of OPN with the organic components of this tissue, and/or from slight variations in the biogenic mineral phase. Most importantly, the crosslinking of OPN occurring as a result of addition of either GTA or TG2 substantially improves the adhesive performance of OPN on both substrates (hydroxyapatite and dentin). By comparing the performances of TG2- and GTA-crosslinked OPN, it appears that the extent of crosslinking of the protein is an important factor for mechanical performance. The results also confirm that blocking TG2 enzymatic activity by adding the inhibitor NC9 effectively cancels the adhesive performance of OPN. Pre-coating of the substrates with OPN further improves the adhesive performances of the samples, likely by allowing monomeric OPN to initially bind to the mineral and matrix components, with this initial attachment providing a protein layer for additional crosslinking between the substrates. The samples and loading used were highly controlled in terms of geometry and scale, which provided the first estimate of the “engineering” fracture toughness of OPN. While this configuration is highly idealized, the toughness reported here reflects adhesive properties and contributions of monomer and polymer (crosslinked) OPN, and mechanisms of adhesion of OPN on mineral. In this sense, the results provided herein bring new insight into the adhesive behavior of OPN in the context of toughening mechanisms that might occur in bone across different length scales.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

[1] Weiner S, Wagner H D. The material bone: Structure mechanical function relations. Annual Review of Materials Science 1998; 28:271-98.
[2] Reznikov N, Shahar R, Weiner S. Bone hierarchical structure in three dimensions. Acta Biomaterialia 2014; 10:3815-26.
[3] Reznikov N, Steele J A M, Fratzl P, Stevens M M. A materials science vision of extracellular matrix mineralization. Nature Reviews Materials 2016; 1.
[4] Barthelat F, Yin Z, Buehler M J. Structure and mechanics of interfaces in biological materials. Nature Reviews Materials 2016; 1.
[5] Bosshardt D D, Zalzal S, McKee M D, Nanci A. Developmental appearance and distribution of bone sialoprotein and osteopontin in human and rat cementum. Anat Rec 1998; 250:13-33.
[6] Fantner G E, Adams J, Turner P, Thurner P J, Fisher L W, Hansma P K. Nanoscale ion mediated networks in bone: Osteopontin can repeatedly dissipate large amounts of energy. Nano Letters 2007; 7:2491-8.
[7] McKee M D, Pedraza C E, Kaartinen M T. Osteopontin and wound healing in bone. Cells Tissues Organs 2011; 194:313-9.
[8] McKee M D, Nanci A. Osteopontin at mineralized tissue interfaces in bone, teeth, and osseointegrated implants: Ultrastructural distribution and implications for mineralized tissue formation, turnover, and repair. Microscopy Research and Technique 1996; 33:141-64.
[9] Gupta H S, Seto J, Wagermaier W, Zaslansky P, Boesecke P, Fratzl P. Cooperative deformation of mineral and collagen in bone at the nanoscale. Proceedings of the National Academy of Sciences of the United States of America 2006; 103:17741-6.
[10] Ager J W, Balooch G, Ritchie R O. Fracture, aging, and disease in bone. Journal of Materials Research 2006; 21:1878-92.
[11] Ritchie R O, Buehler M J, Hansma P. Plasticity and toughness in bone. Phys Today 2009; 62:41-7.
[12] Christensen B, Zachariae E D, Scavenius C, Kloverpris S, Oxvig C, Petersen S V, et al. Transglutaminase 2-catalyzed intramolecular cross-linking of osteopontin. Biochemistry 2016;55:294-303.
[13] Butler W T. Structural and functional domains of osteopontin. In: Denhardt D T, Butler W T, Chambers A F, Senger D R, editors. Osteopontin: Role in Cell Signalling and Adhesion1995. p. 6-11.
[14] Sorensen E S, Rasmussen L K, Moller L, Jensen P H, Hojrup P, Petersen T E. Localization of transglutaminase-reactive glutamine residues in bovine osteopontin. Biochemical Journal 1994;304:13-6.
[15] Boukpessi T, Hoac B, Coyac B R, Leger T, Garcia C, Wicart P, et al. Osteoporitin and the dento-osseous pathobiology of X-linked hypophosphatemia. Bone 2017; 95:151-61.
[16] Denda S, Reichardt L F, Muller U. Identification of osteopontin as a novel ligand for the integrin alpha 8 beta 1 and potential roles for this integrin-ligand interaction in kidney morphogenesis. Molecular Biology of the Cell 1998; 9:1425-35.
[17] Thurner P J, Chen C G, Ionova-Martin S, Sun L L, Harman A, Porter A, et al. Osteopontin deficiency increases bone fragility but preserves bone mass. Bone 2010; 46:1564-73.
[18] Poundarik A A, Diab T, Sroga G E, Ural A, Boskey A L, Gundberg C M, et al. Dilatational band formation in bone. Proceedings of the National Academy of Sciences of the United States of America 2012; 109:19178-83.
[19] Siegel R C, Pinnell S R, Martin G R. Cross-linking of collagen and elastin—Properties of lysyl oxidase. Biochemistry 1970; 9:4486-92.
[20] Siegel R C. Collagen cross-linking. Synthesis of collagen cross-links in vitro with highly purified lysyl oxidase. Journal of Biological Chemistry 1976; 251:5786-92.
[21] Kaartinen M T, El-Maadawy S, Rasanen N H, McKee M D. Tissue transglutaminase and its substrates in bone. J Bone Miner Res 2002; 17:2161-73.
[22] Wang S, Kaartinen M T. Cellular factor XIIIA transglutaminase localizes in caveolae and regulates caveolin-1 phosphorylation, homo-oligomerization and c-Src signaling in osteoblasts. J Histochem Cytochem 2015; 63:829-41.
[23] Kaartinen M T, Pirhonen A, Linnala-Kankkunen A, Maenpaa P H. Cross-linking of osteopontin by tissue transglutaminase increases its collagen binding properties. Journal of Biological Chemistry 1999; 274:1729-35.
[24] Aeschlimann D, Kaupp O, Paulsson M. Transglutaminase-catalyzed matrix cross-linking in differentiating cartilage: Identification of osteonectin as a major glutaminyl substrate. Journal of Cell Biology 1995; 129:881-92.
[25] Jurgensen K, Aeschlimann D, Cavin V, Genge M, Hunziker E B. A new biological glue for cartilage-cartilage interfaces: Tissue transglutaminase. Journal of Bone and Joint Surgery-American Volume 1997; 79A:185-93.
[26] Griffiths G. Fine Structure Immunocytochemistry. In: Springer-Verlag, editor. Berlin, Germanyl993. p. 29-36.
[27] Wine Y, Cohen-Hadar N, Freeman A, Frolow F. Elucidation of the mechanism and end products of glutaraldehyde crosslinking reaction by X-ray structure analysis. Biotechnology and Bioengineering 2007; 98:711-8.
[28] McKee M D, Nanci A. Secretion of osteopontin by macrophages and its accumulation at tissue surfaces during wound healing in mineralized tissues: A potential requirement for macrophage adhesion and phagocytosis. Anat Rec 1996; 245:394-409.
[29] McKee M D, Nanci A. Postembedding colloidal-gold immunocytochemistry of noncollagenous extracellular matrix protein in mineralized tissues. Microscopy Research and Technique 1995; 31:44-62.
[30] Sorensen E S, Hojrup P, Petersen T E. Posttranslational modifications of bovine osteopontin: Identification of twenty-eight phosphorylation and three O-glycosylation sites. Protein Science 1995; 4:2040-9.
[31] Sorensen E S, Petersen T E. Purification and characterization of 3 proteins isolated from the proteose peptone fraction of bovine-milk. Journal of Dairy Research 1993; 60:189-97.
[32] Caron N S, Munsie L N, Keillor J W, Truant R. Using FLIM-FRET to measure conformational changes of transglutaminase type 2 in live cells. Plos One 2012;7.
[33] Tjaderhane L, Carrilho M R, Breschi L, R. Tay F R, Pashley D H. Dentin basic structure and composition—an overview. Endodontic Topic 2012; 20:3-29.
[34] Dastjerdi A K, Tan E, Barthelat F. Direct measurement of the cohesive law of adhesives using a rigid double cantilever beam technique. Experimental Mechanics 2013; 53:1763-72.
[35] Dastjerdi A K, Pagano M, Kaartinen M T, McKee M D, Barthelat F. Cohesive behavior of soft biological adhesives: Experiments and modeling. Acta Biomaterialia 2012; 8:3349-59.
[36] Liao C-H, Watson B, Worswick M J, Cronin D S. Mode I Rigid Double Cantilever Beam Test and Analysis Applied to Structural Adhesives. In: Kimberley J, Lamberson L, Mates S, editors. Dynamic Behavior of Materials, Volume 1: Proceedings of the 2017 Annual Conference on Experimental and Applied Mechanics. Cham: Springer International Publishing; 2018. p. 73-81.
[37] Higashikawa F, Eboshida A, Yokosaki Y. Enhanced biological activity of polymeric osteopontin. Febs Letters 2007; 581:2697-701.
[38] Aeschlimann D, Thomazy V. Protein crosslinking in assembly and remodelling of extracellular matrices: The role of transglutaminases. Connective Tissue Research 2000; 41:1-27.
[39] Goldsmith H L, Labrosse J M, McIntosh F A, Maenpaa P H, Kaartinen M T, McKee M D. Homotypic interactions of soluble and immobilized osteopontin. Annals of Biomedical Engineering 2002; 30:840-50.
[40] Addison W N, Masica D L, Gray J J, McKee M D. Phosphorylation-Dependent Inhibition of Mineralization by Osteopontin ASARM Peptides Is Regulated by PHEX Cleavage. J Bone Miner Res 2010; 25:695-705.
[41] Addison W N, Azari F, Sorensen E S, Kaartinen M T, McKee M D. Pyrophosphate inhibits mineralization of osteoblast cultures by binding to mineral, up-regulating osteopontin, and inhibiting alkaline phosphatase activity. Journal of Biological Chemistry 2007; 282:15872-83.
[42] Hoac B, Nelea V, Jiang W G, Kaartinen M T, McKee M D. Mineralization-inhibiting effects of transglutaminase-crosslinked polymeric osteopontin. Bone 2017; 101:37-48.
[43] Christensen B, Zachariae E D, Scavenius C, Thybo M, Callesen M M, Kløverpris S, et al. Identification of transglutaminase reactive residues in human osteopontin and their role in polymerization. Plos One 2014; 9.
[44] Migneault I, Dartiguenave C, Bertrand M J, Waldron K C. Glutaraldehyde: behavior in aqueous solution, reaction with proteins, and application to enzyme crosslinking. Biotechniques 2004;37:790-6, 8-802.
[45] McKee M D, Zalzal S and Nanci A (1996) Extracellular matrix in tooth cementum and mantle dentin: Localization of osteopontin and other noncollagenous proteins, plasma proteins and glycoconjugates by electron microscopy. Anat. Rec. 245:293-312.
[46] McKee M D, and Nanci A (1996) Osteopontin at mineralized tissue interfaces in bone, teeth and osseointegrated implants: Ultrastructural distribution and implications for mineralized tissue formation, turnover and repair. Microsc. Res. Tech. 33:141-164

What is claimed is:

1. A method of treating an exposed surface of a mineralized tissue, the method comprising contacting a mineralized tissue adhesive or filler composition having intermolecular crosslinked osteopontin (OPN) protein directly with (i) the exposed surface or (ii) a first layer of monodisperse OPN protein directly deposited on the exposed surface so as to treat the exposed surface.

2. The method of claim 1, wherein the exposed surface comprises at least one crack and the method further comprises filing the at least one crack.

3. The method of claim 1, further comprising contacting the mineralized tissue adhesive or filler composition directly with (i) a second surface or (ii) a second layer of monodisperse OPN protein directly deposited on the second surface so as to adhere the first surface to the second surface.

4. The method of claim 3, wherein the second surface is a mineralized tissue.

5. The method of claim 3, wherein the second surface is an artificial surface.

6. The method of claim 1, further comprising depositing monodisperse OPN protein on the exposed surface to form the first layer of monodisperse OPN protein.

7. The method of claim 1, further comprising providing the mineralized tissue adhesive or filler composition.

8. The method of claim 7, comprising cross-linking monomeric OPN proteins with a crosslinking agent to provide the mineralized tissue adhesive or filler composition.

9. The method of claim 8, wherein the crosslinking agent is a transglutaminase.

10. The method of claim 9, wherein the transglutaminase is transglutaminase 2.

11. The method of claim 8, wherein the crosslinking agent is glutaraldehyde.

12. The method of claim 1, wherein the mineralized tissue is a tooth.

13. The method of claim 12, wherein the exposed surface comprises enamel, dentin and/or cementum.

14. The method of claim 1, wherein the mineralized tissue is in a subject.

15. A kit for making and using a mineralized tissue adhesive or filler composition to treat an exposed surface of a mineralized tissue, the kit comprising:
(a) a monodisperse osteopontin (OPN) protein composition;
(b) a crosslinking agent; and
(c) instructions for:
preparing the mineralized tissue adhesive or filler composition by cross-linking, with the cross-linking agent, the monodisperse OPN protein composition, wherein the mineralized tissue adhesive or filler composition comprises intermolecular crosslinked OPN protein;
optionally preparing a monodisperse solution of OPN protein from the monodisperse OPN protein composition;
optionally depositing a first layer of monodisperse OPN protein on the exposed surface of the mineralized tissue to form a first layer of monodisperse OPN protein;
optionally depositing a second layer of monodisperse OPN protein on a second surface to form a second layer of monodisperse OPN protein; and
directly contacting the mineralized tissue adhesive or composition on (i) the exposed surface of the mineralized tissue or on the first layer of monodisperse OPN protein and optionally on (ii) the second surface or on the second layer of monodisperse OPN protein.

16. A dental protective coating comprising the mineralized tissue adhesive or filler composition as defined in claim 1.

17. A dental filling comprising the mineralized tissue adhesive or filler composition as defined in claim 1.

18. A dental adhesive comprising the mineralized tissue adhesive or filler composition as defined in claim 1.

19. The method of claim 1, wherein the mineralized tissue is a bone.
